(19) United States
(12) Patent Application Publication
(10) Pub. No.: US 2020/0337297 A1
(43) Pub. Date: Oct. 29, 2020

(54) SYSTEM AND METHOD FOR CARBON MONOXIDE ATMOSPHERE STORED BLOOD COMPONENTS

(71) Applicant: SAFETIN LTD., TEL AVIV (IL)

(72) Inventors: NURITH SHAKLAI, TEL AVIV (IL); MATITYAHU SHAKLAI, TEL AVIV (IL)

(21) Appl. No.: 16/764,866

(22) PCT Filed: Nov. 16, 2018
(86) PCT No.: PCT/IL2018/051237

§ 371 Date: May 17, 2020

Related U.S. Application Data
(60) Provisional application No. 62/586,926, filed on Nov. 16, 2017.

(51) Int. Cl. A01N 1/02; C12M 1/00; A61J 1/05; A61M 1/02

(52) U.S. Cl. CPC A01N 1/0242; A01N 1/021; C12M 47/04; C12M 31/00; A61M 2205/18; A61M 1/0272; A61M 2202/0233; A61M 2202/0291; A61M 2202/0427; A61J 1/05

(57) ABSTRACT

A system, apparatus, and method using carbon monoxide for treatment of platelets, and optionally other blood components and/or whole blood, allowing extended storage and increased viability of the stored blood platelets in vivo. The system allows an efficient use of the stored platelets by dividing the platelets into a population with a short residual lifetime for immediate use and a population that can remain in storage for longer. The storage apparatus enables safe platelet preservation and is equipped with an alarm to detect CO leakage.

### FIELD OF DISCLOSURE

The disclosure relates to storage of blood products and more specifically to storage of platelets in carbon monoxide (CO) atmosphere, preferably completely devoid of oxygen.

BACKGROUND OF THE DISCLOSURE

Blood transfusion is a central therapeutic aid in modern medicine and is a primary treatment in the field of emergency medicine. The main obstacle to blood collection for transfusion is that blood is an untradeable material which can be obtained by donation only thereby limiting the total amount collected. For this reason, since the beginning of the twentieth century, blood has been collected and stored in blood banks. Initially blood was stored as whole blood, but today it is separated into defined components before storage for patient treatment. These blood components are stored in closed plastic bags at temperatures ranging from −80° C. to +24° C., depending upon the particular blood component.

Although human blood is distributed internationally, maintaining an adequate supply depends upon a number of factors: the availability of donors, the provision of suitable collection, storage facilities, and the limited shelf-life. Therefore, the medical community is interested in developing new procedures for extending the shelf-life of blood components.

Theoretically, the shelf-life of preserved blood components depends upon two major factors: the time period during which the function of the blood components can be maintained in storage and the reduction of pathogen contamination. The extended maintenance of blood component function in storage has been achieved by adding such materials as phosphates and/or other compounds to arrest undesirable biological activity such as coagulation, changing the pH balance of the storage medium and maintaining the proper temperature for the particular component. For certain types of blood components reduced temperature levels are suitable for storage and also help to reduce the rate of the growth of contaminating microorganisms. However, for other components, such as platelets, reduced temperature may induce a loss of biological function, and therefore cannot be used to reduce pathogen contamination.

Contamination of blood by pathogens has long been recognized as a significant complication of blood transfusion. Even if healthy donors are selected and the resultant donated blood is screened for the presence of various types of pathogens, including viruses such as hepatitis and HIV, blood components which are stored for an extended period of time are vulnerable to pathogen contamination.

In order to reduce contaminations, blood is collected from donors under aseptic conditions. Sterile closed systems are used for the collection and processing of blood components, further reducing pathogen contamination. However, the presence of bacteria in blood components is still currently the most common microbiological cause of transfusion-associated morbidity and mortality. Transfusion-associated contamination which is caused by the inadvertent intravenous infusion of pathogen contaminated platelets appears to be much more common than complications caused by contamination of red blood cells or plasma. This may be due to the fact that significant morbidity and mortality occurs when the contaminated blood product contains a sufficiently large number of bacteria, thereby resulting in a relatively high level of bacterial endotoxins. Since platelets currently cannot be stored at temperatures lower than 20° C. without risking the loss of biological function, the risk of contamination is proportionally much larger with platelets than with red blood cells. Indeed, the rate of reported complications from infected platelets is greater than that of red blood cells by a 2:1 ratio.

Platelets are enucleated cells derived from bone marrow megakaryocytes. They play an important role in hemostasis, blood clotting and thrombosis. The life span for platelets in blood circulation is estimated to be about ten to twelve days. However, after five to six days of ex-vivo storage, platelets age, as evidenced by morphological signs of apoptosis such as a change in shape from discoid to spherical, and the presence of membrane blabbing. Another measurable parameter for platelet viability is the pH of the surrounding medium; when it falls below pH 6.0, viability is lost. An additional measurement of platelet viability is the leakage of enzymes, e.g. LDH (lactic dehydrogenase).

Various studies have confirmed that pathogen contamination of platelets causes the highest level of mortality of all the different blood components and products. For allogeneic transfusions, the mortality rate for apheresis platelet transfusion was seven times higher than the risk of an adverse event following platelet concentrate transfusion, and more than three times higher than the risk following red blood cell transfusion. The risk increases to twelve times higher after platelet pool transfusion (from multiple donors) and 5.5 times higher after apheresis platelet concentrate infusion (all statistics from Perez P, Salmi L R et al., “ Determinants of transfusion-associated bacterial contamination: results of the French BACTHEM Case-Control Study”, Transfusion 2001, 41:862-872; see also Sazama K, “Bacteria in Blood for Transfusion. A Review.”, Arch Pathol Lab Med 1994, 118:350-365). These studies of the risk of platelet contamination have led to the shortening of allowed platelet storage from 7 to 5 days by the FDA in 1986, significantly reducing the available supplies of platelets.

The medical community is currently considering two options: 1) providing blood banks with more rapid bacterial screening methods and 2) developing methods for the control of growth of bacteria and/or other pathogens. The former approach has a number of drawbacks, including lower sensitivity of the more rapid bacterial detection methods and increased expense. The latter approach has been explored generally involving the destruction of the ability to replicate genetic material, as this approach is believed to be safe for enucleated blood cells like red cells and platelets. For example, cross-linking chemicals, with and without the requirement for photo activation are in use. Other materials in use include psoralens 8-MOP and AMT. These chemicals are considered to be hazardous to the human body and thus must be removed post-treatment, before the platelets can be transfused. Current removal methods include filtration or washing protocols in order to remove agents which are not bound in some manner to the surface of cells or proteins. Since the removal process is time consuming and may also damage the blood cells, other less hazardous agents have been considered. An example is riboflavin, which upon photo activation forms lumichrome. However, this agent has been shown to have variable effectiveness for bacterial inactivation and may even decrease platelet survival rates in autologous transfusions performed in primates, which has negative implications for its utility in promoting increased platelet storage times (“Connect with Safer Blood Products: Abstracts on Pathogen Eradication Technology”, Gambro BCT Inc., USA, 2001).

Of the methods described, each one has a number of disadvantages leading to reduced lifetime of the transfused platelets in circulation, as well as decrease in platelet function. Moreover, currently there is no suitable method for preservation of platelets which does not involve introduction of potentially hazardous chemicals into the human body. The background art does not teach or suggest an effective method for storage of platelets, which is readily reversible and which does not cause permanent damage or alteration to any part of the platelets. The background art also does not teach or suggest a method in which a relatively non-toxic agent, which can also be removed prior to infusion, is used for platelet storage.

This situation provides an urgent need to find a suitable method and system for extending the time that platelets can be preserved, that does not pose any threat from use of potentially hazardous chemicals, which does not damage the stored platelets, and which preferably increases the yield of platelets. It would be desirable to find preservative material that exists in a gas state at room temperature that could be easily removed prior to transfusion of the stored platelets. Carbon monoxide (CO) is a natural gas product of hemoproteins degradation in the mammalian organism and practically chemically inert. It has been known as a highly toxic gas due to its ability to replace, with high affinity, the sites for oxygen in hemoglobin. However, a growing body of scientific evidence has indicated that the same molecule serves also basic physiological roles like neurotransmission. Thus, its location and quantity appears to determine whether carbon monoxide is helpful or harmful to the body. CO has been shown to prevent peroxidative damage derived from the combined presence of iron and oxygen peroxide (Sher E A, Shaklai M, Shaklai N. Carbon monoxide promotes respiratory hemoproteins iron reduction using peroxides as electron donors. PLoS One. 2012 Mar. 12; 7(3):e33039). Moreover, scientific literature indicates that CO has anti-aggregatory effect on Plt (Brüne B, Ullrich V O. Inhibition of platelet aggregation by carbon monoxide is mediated by activation of guanylate cyclase. Molecular Pharmacology. 1987 Oct. 1; 32(4):497-504).

U.S. Pat. No. 7,323,295, by the present inventors and owned in common with the present application, relates to the use of CO treatment of whole blood and/or blood components to increase cell viability after storage. CO, in the small amounts left in platelets concentrate (PC) after exposure to air is sufficiently non-toxic to be tolerated by the body and, as above, is known nowadays as a metabolic component. However, the high concentrations of CO gas needed for the process are toxic and are not detectable through smell, thereby posing an environmental threat. Further, this disclosure is lacking a method to increase the yield of platelets.

There is therefore an urgent need to find a suitable method and system for extending the time that platelets can be preserved, that does not pose any threat from use of potentially hazardous chemicals, which does not damage the stored platelets, and which increases the yield of platelets.

### SUMMARY OF THE DISCLOSURE

The present disclosure overcomes deficiencies of the background art by providing a system, method and device for extending the storage period of platelets by treatment of platelets with carbon monoxide (CO), followed by separation of platelets into different populations. The present disclosure, in at least some embodiments, relates to improving platelets storage, through storage of the platelets under an oxygen free, carbon monoxide atmosphere (COatm) using a dedicated storage device. Optionally the platelets are separated from COatm-treated whole blood.

According to some embodiments, the method also increases effective utilization of platelets by separation of the platelets into two populations by phosphatidylserine (PS) exposure on the outer cell membrane. Preferably, the method also increases utilization of collected platelets by separating them according to thrombogenic activity.

According to some embodiments, the cells in at least one blood component and/or whole blood are separated into a plurality of populations according to at least one marker, which preferably correlates with different characteristics. More preferably, the characteristic correlates with the length of storage time that the cells are expected to remain viable. Optionally, the characteristic is determined, and the cells separated after exposure to an oxygen-containing atmosphere before being administered to a subject.

According to at least some embodiments, for increased safety, a platelet storage device is provided with nested containers: A core storage container with an atmosphere of CO (COatm) which extends the platelet shelf-life and inhibits pathogen proliferation; and an outer storage container within which this core storage container is placed for protecting the device vicinity from undesired CO leakage. Optionally the outer storage container includes an additional neutral gas (such as N₂). Preferably, the outer storage container comprises an alarm that indicates detection of leaked CO such as by changing color.

As demonstrated previously (U.S. Pat. No. 7,323,295), the present disclosure also provides a method for inhibiting bacterial growth in whole blood and/or blood components, which may therefore also be used to extend the storage time for whole blood and/or blood components, through treatment with CO. According to a preferred embodiment of the present disclosure, donated whole blood is first separated into various components, after which, more preferably, only the platelet fraction is treated with CO. Alternatively, donated whole blood is treated with CO, after which more preferably the platelet fraction is treated again with CO. Alternatively or additionally, for either embodiment, the plasma fraction may also optionally be treated with CO. Whole blood which has been treated with CO may also optionally be used for transfusion after gas exchange by air.

The method of treatment according to the present disclosure more preferably includes removing air from the container which holds the platelet or any other fraction, and then introducing CO as the only gas component, thereby creating an inert gas atmosphere that excludes oxygen. Minor components of the anaerobic atmosphere might include inert gases other than CO, for example, xenon that has already been shown to serve as an atmosphere for extending blood cell storage. Moreover, an inert gas like xenon allows storage of platelets under elevated pressure

and/or reduced temperature while maintaining their function (U.S. Pat. No. 8,652,770B2). Besides inhibition of the pathogens growth and extension of the shelf-life of platelets and of whole blood, the separation of the cells into different populations further increases the yield, specifically for platelets (Plt) with a short life time. Platelets with a high PS exposure are hyperactive but with a short shelf-life and are preferably used for immediate transfusion. The remaining platelets can be stored for a further extended period of time, while still maintaining their activity potential, thereby increasing the percentage of active platelets that are administered to a subject.

Although reference is made to treatment of blood components, this is for the purposes of explanation only and is not meant to be limiting in any way, as the present disclosure is also suitable for the treatment of whole blood. Hereinafter, the term “blood product” refers to at least one of whole blood and/or a blood component, such as platelets for example.

According to some embodiments of the present disclosure a platelet storage device comprises: a core container adapted for extended storage of Platelet, wherein the adaptation comprises a COatm inside the core container, wherein the core container is adapted to be closed so that it is gas impermeable; and an outer container for storing the core container, wherein the outer container is adapted to be closed so that it is gas impermeable. Preferably the COatm comprises carbon monoxide as a major component. Optionally the COatm comprises up to 100% carbon monoxide.

Optionally the COatm further comprises another gas such as xenon. Optionally the temperature is in the range of 20-24° C. Optionally the outer container comprises an inert gas. Optionally the outer container comprises a CO alarm.

According to similar embodiments of the present disclosure a method of storing platelets using the storage device as disclosed above comprises: inserting platelets into the core container; treating the platelets by replacing air in the core container with CO and sealing the core container; placing the core container into the outer container; and sealing the outer container. Optionally the method further comprises: opening the outer container; opening the core container to allow escape of the COatm; and illuminating the platelets with a light source for removal of attached CO.

Optionally the method further comprises: filling the outer container with an inert gas before sealing the outer container; extracting the platelets from the core container; dividing the extracted platelets into two populations according to at least one characteristic of the platelets correlating with expected viability after further storage, long viability platelets and short viability platelets; for the long viability platelets: inserting the long viability platelets into the core container; replacing air in the core container with CO and sealing the core container; placing the core container into the outer container; and sealing the outer container; and for the short viability platelets: immediately using the short viability platelets for transfusion in a patient or optionally for the short viability platelets: storing the short viability platelets in short-term storage.

Optionally the method further comprises the step of storing the platelets in the storage device at a suitable temperature, wherein viability of the stored platelets is retained. Optionally the treating step further comprises adding a pH buffering substance to the platelets. Optionally the pH buffering substance comprises bicarbonate.

According to further embodiments of the present disclosure a method for increasing the yield of stored platelets comprises: storing the platelets in a COatm; and extracting the platelets and separating the platelets into a plurality of populations according to at least one characteristic correlating with expected viability after further storage, long viability platelets and short viability platelets. Preferably the at least one characteristic relates to PS exposure, such that increased extent of PS exposure correlates with reduced expected viability after further storage. Optionally the separating is performed by a FACS machine.

Preferably the method further comprises selecting the short viability platelet population for immediate or rapid use according to the increased extent of PS exposure; and selecting the long viability platelet population for further storage in COatm.

According to further embodiments of the present disclosure a platelet storage device adapted for extended storage of platelets under oxygen free CO atmosphere and for preventing toxicity from a potential CO leakage comprises: a core container adapted for extended storage of platelets, wherein the adaptation comprises a COatm inside the core container, wherein the core container is adapted to be closed so that it is gas impermeable; and an outer container for storing the core container, wherein the outer container is adapted to be closed so that it is gas impermeable, wherein the outer container comprises a CO alarm.

The terms platelet, platelet concentrate (PC), and Plt are used interchangeably herein. Optionally the term cells as used herein refers to platelets. Platelets optionally include at least one of PRP and PC fractions. The term COatm as used herein refers to an oxygen free, carbon monoxide atmosphere where the atmosphere is of the gas in an enclosed container.

Implementation of the method and system of the present disclosure involves performing or completing certain selected tasks or steps manually, automatically, or a combination thereof. Moreover, according to actual instrumentation and equipment of preferred embodiments of the method and system of the present disclosure, several selected steps could be implemented by hardware or by software on any operating system of any firmware or a combination thereof.

### BRIEF DESCRIPTION OF THE DRAWINGS

The foregoing and other aspects and advantages will be better understood from the following detailed description of a preferred embodiment of the disclosure with reference to the drawings, wherein:

FIGS. 1A-1D show schematic drawings of devices for blood component separation and storage and 1E shows a process for storage of blood components according to some embodiments of the present disclosure;

FIG. 2 shows an exemplary system incorporating the apparatus of FIGS. 1A-1D, according to at least some embodiments of the present disclosure;

FIG. 3A is a flow diagram of a method for separation of a specific blood component into a plurality of populations and 3B shows an exemplary output from a FACS analysis according to some embodiments of the present disclosure;

FIG. 4 shows a comparison of the ATP levels in Plt stored as PC under: air, COatm, as well as re-aerated platelets following COatm storage;

FIG. 5 shows a comparison of in vivo survival of COatm and air-stored human platelets in a rabbit circulation model; and

FIG. 6 shows a comparison of PS exposure of fresh platelets and those stored under Air or COatm after specific periods of time.

DESCRIPTION OF PREFERRED EMBODIMENTS

Headings are included herein to aid in locating certain sections of detailed description. These headings should not be considered to limit the scope of the concepts or embodiments described under any specific heading. Furthermore, concepts or embodiments described in any specific heading are generally applicable in other sections or may optionally be combined with other sections throughout the entire specification.

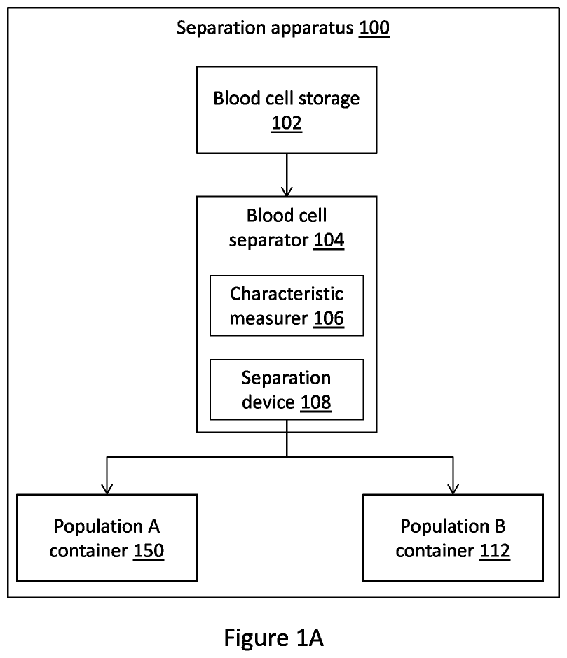

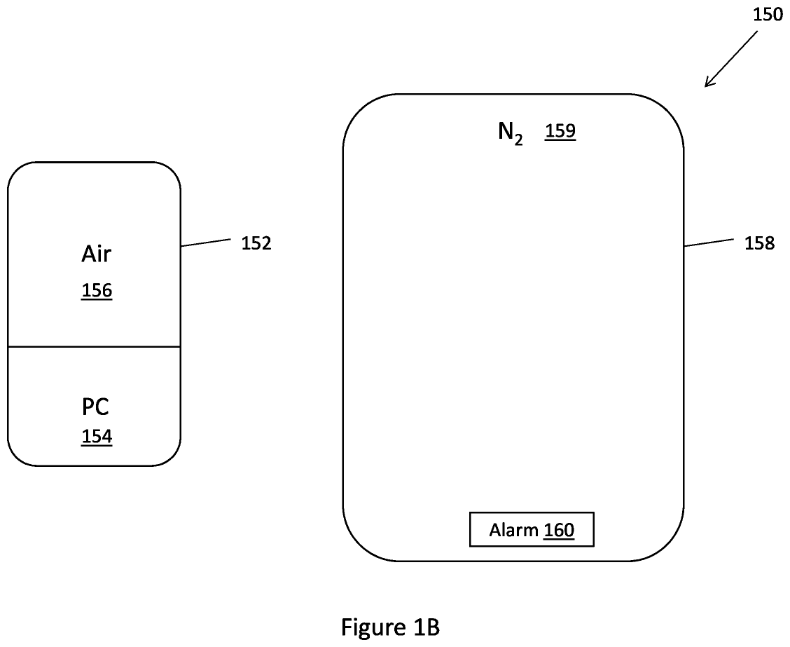

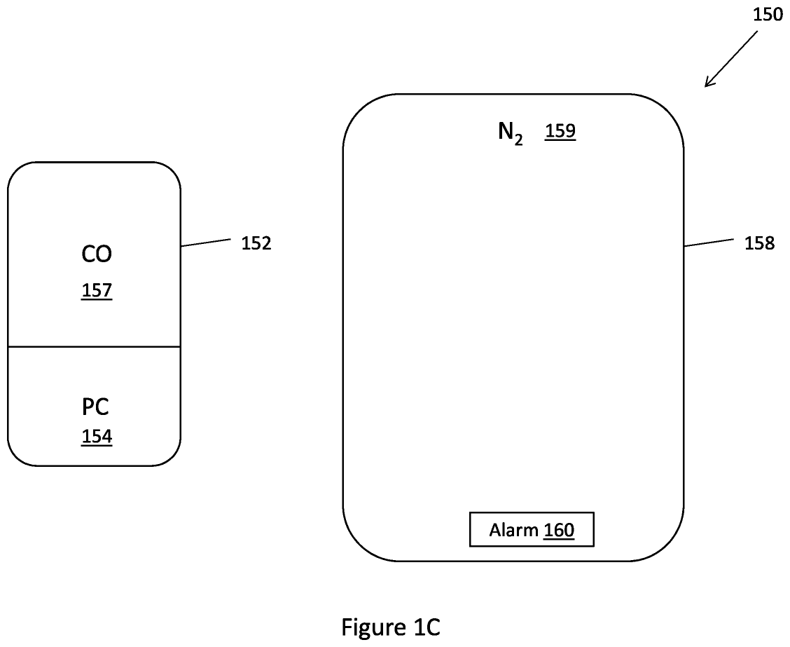

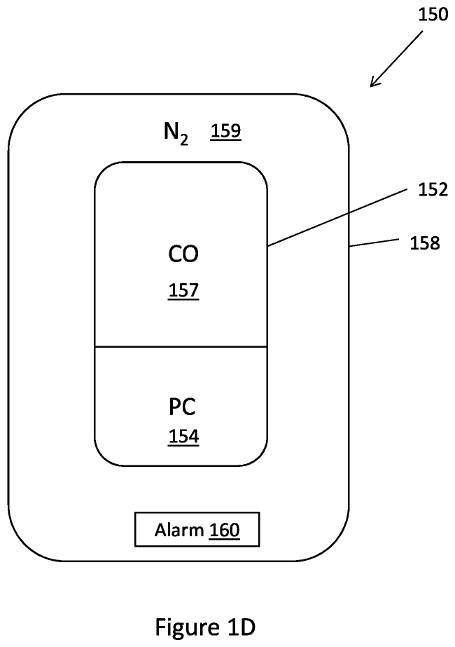

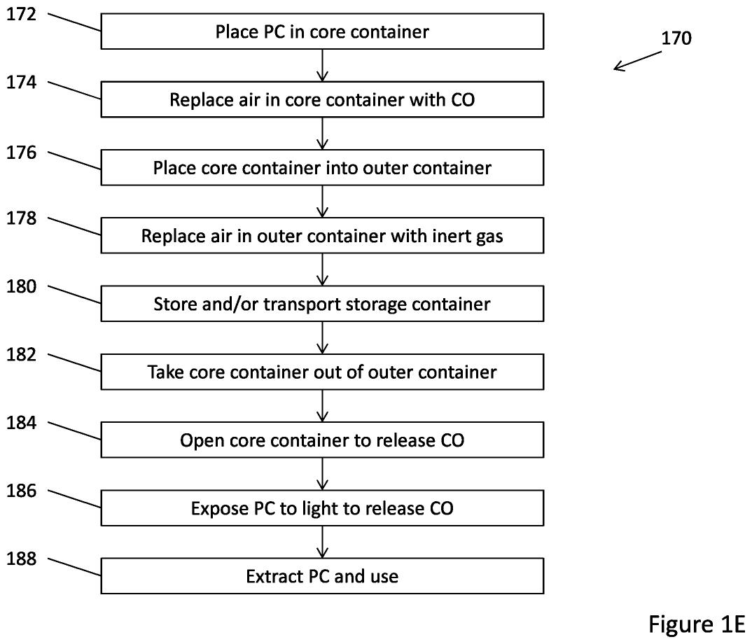

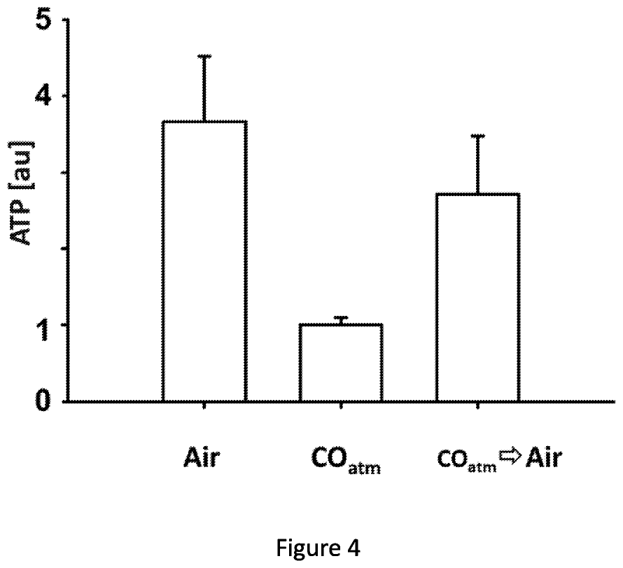

The present disclosure is of a system, method and device for extending the storage period of platelets by treatment of platelets with carbon monoxide (CO) and preventing exposure of the platelets to oxygen, followed by separation of platelets into different populations. FIGS. 1A-1E, 2 and 3 show exemplary embodiments of systems and methods for storage and separation of platelets into different populations. FIGS. 4-6 and the accompanying descriptions illustrate experimental proof that forms the basis of the systems and methods disclosed.

Reference is now made to FIGS. 1A-1D showing schematic drawings of devices for blood component separation and storage and 1E showing a process for storage of blood components according to some embodiments of the present disclosure. As shown in FIG. 1A, a separation apparatus 100 features a blood cell storage 102 for holding or receiving cells to be separated. Blood cell storage 102 is optionally storage device 150 as described further below. The cells may optionally already be separated into different blood components for example, such as platelets and other blood components. Alternatively, the cells may optionally be in the form of whole blood. A blood cell separator 104 receives the cells to be separated from blood cell storage 102.

Blood cell separator 104 separates the cells into a plurality of different populations according to expected viability after a further period of storage. Preferably, this is accomplished according to at least one characteristic of the cell populations. This characteristic is preferably measured by a characteristic measurer 106, according to which separation is determined. Separation is then preferably performed by a separation device 108. Optionally, blood cell separator 104 is a fluorescence activated cell sorter (FACS) machine, for example.

After separation, the various cell populations are optionally sent to different containers, such as for example a population A storage device 150 and a population B container 112.

As shown (FIGS. 1B-1D), storage device 150 comprises core container 152, which may optionally be a plastic sac for example. Core container 152 is adapted to be sealed so that it is gas impermeable to prevent the release of CO 157. Platelets as concentrate (PC) 154 are placed into core container 152 for storage, initially in an air atmosphere 156 (FIG. 1A). The air 156 is replaced by CO 157 (FIG. 1B). Core container 152 is then placed in an outer container 158 (FIG. 1C), containing an inert gas 159 such as N₂ for example. Outer container 158 may optionally be a plastic sac for example. Outer container 158 is adapted to be sealed so that it is gas impermeable to prevent the release of inert gas 159. Optionally outer container 158 also includes a CO alarm 160, which may optionally comprise a material that changes color after exposure to CO or may provide another alarm such as an audible alarm. Core container 152 and outer container 158 comprise opening and sealing mechanisms, piping, valves and other gas and fluid inflow and outflow mechanisms as required.

FIG. 1E shows a process 170 for storing PC 154 using storage container 150 according to some embodiments of the present disclosure. In step 172 PC 154 is placed into core container 152 which also contains air 156. In step 174 the air 156 in core container 152 is replaced with CO 157 and core container 152 is sealed so as to be gas impermeable. In step 176, core container 152 is placed into outer container 158. In step 178 the air in outer container 158 is optionally replaced with an inert gas 159 and outer container 158 is sealed so as to be gas impermeable. If required, optionally alarm 160 is activated. In step 180 container 150 is stored or transported. The suitability for PC 154 to be used is optionally determined at this point, for example, according to process 300 described below.

In step 182 when PC 154 is to be used, core container 152 is removed from outer container 158. Optionally outer container 158 is opened and core container 152 is opened while still inside outer container 158. In step 184 core container 152 is opened to release CO, preferably in a well ventilated area. In step 186 the PC 154 is exposed to a light source to cause release of the CO attached to the PC. In step 188 PC 154 is extracted and used as needed.

It should be appreciated that the process of storing and transporting PC is made easy by the sac containers of disclosed storage container 150. It should further be appreciated that extracting PC from the disclosed storage container 150 is also a simple process requiring opening of the container and exposure of the PC to a light source. Unlike prior art methods, no complex washing and detoxification steps are required.

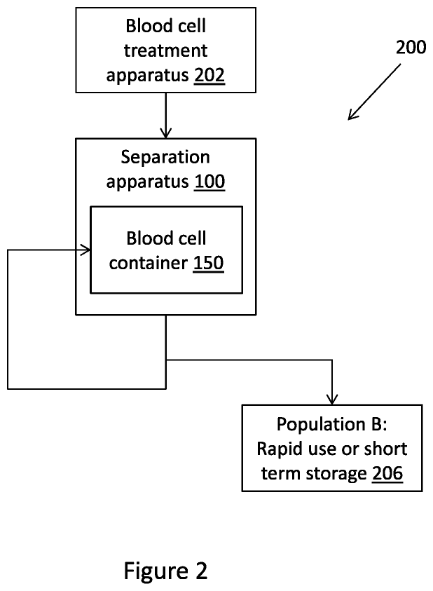

Reference is now made to FIG. 2 which shows an exemplary system incorporating the apparatus of FIGS. 1A-1D, according to at least some embodiments of the present disclosure. In a system 200, optionally the various components are provided in a single machine, a series of connected apparatuses or a plurality of apparatuses that are connected functionally if not physically, or a combination thereof.

As shown, system 200 also features separation apparatus 100 of FIG. 1D, optionally in a different configuration than that shown in FIG. 1D. System 200 also features a blood cell treatment apparatus 202, for example for treating blood cells with CO to increase their storage life. Cells are then preferably stored in blood cell storage 150.

After a period of time in storage 150, the cells are then separated into a plurality of populations, shown as population A and population B, by separation apparatus 100. Population A contains cells that may optionally be stored for an additional period of time. These cells are optionally returned to storage 150 from separation apparatus 100. Population B contains cells that are preferably used more rapidly or even immediately. These cells are optionally sent to a short term storage 206.

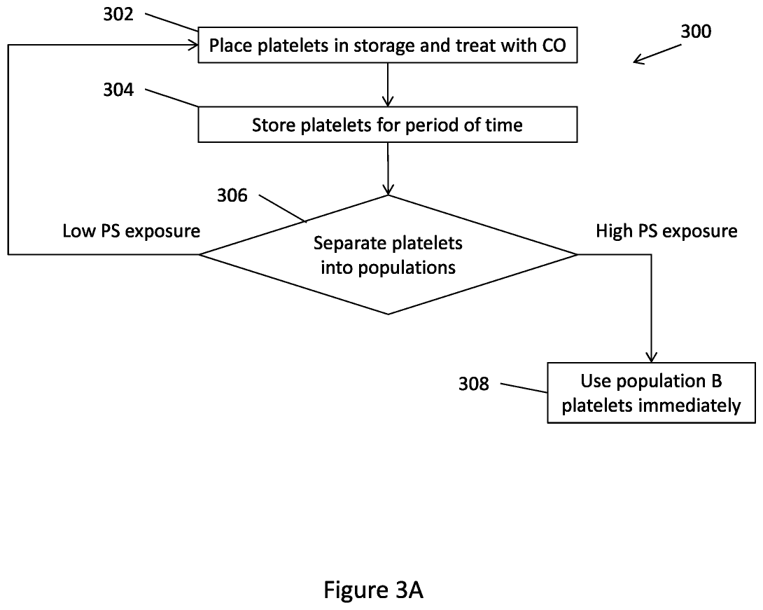

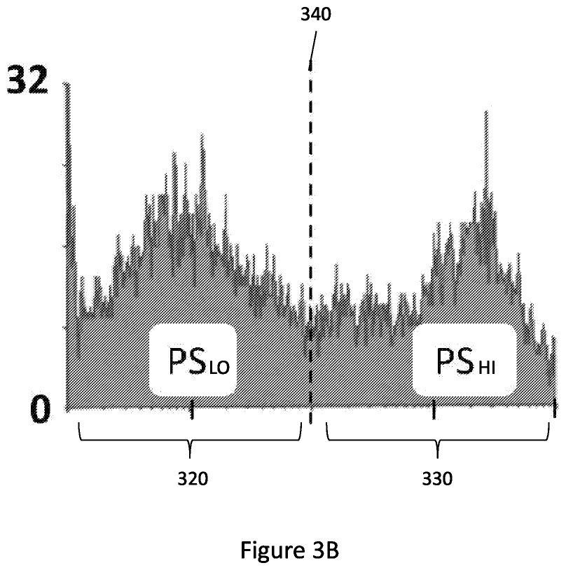

Reference is now made to FIG. 3A which is a flow diagram of a method for separation of a specific blood component into a plurality of populations and 3B which shows an exemplary output from a FACS analysis according to some embodiments of the present disclosure. As shown in process 300, separation of a specific blood component into a plurality of populations is provided. As an example only, the separation is of platelets. In process 300 platelets are separated into a plurality of populations using the apparatus of FIGS. 1A-1D and FIG. 2. In step 302, platelets are placed in a platelet storage device 150 and the atmosphere in the storage 150 is replaced with CO to create a COatm for treatment of the platelets with CO. In step 304, the platelets are stored in COatm in storage device 150 for a period of time.

In step 306, after a period of time in storage 150, platelets are then separated into a plurality of populations, shown as population A and population B, by separation apparatus 100.

The non-limiting, exemplary separation method comprises fluorescence activated cell sorter (FACS) analysis, although any suitable method (or combination of methods) could be used. The non-limiting, exemplary characteristic according to which separation is performed, comprises phosphatidylserine (PS) exposure.

PS is a phospholipid component of membranes which resides in the inner layer of the membrane in all cells. It is now known that apoptosis, as well as variety of stimulations, including platelet activation, leads to exposure to the outer membrane and thus to the cell surface. PS presence on the surface of platelets is a marker of either activity (reversible process) or apoptosis (irreversible).

In either case, platelets with low PS exposure would be expected to be viable after a further extended period of storage (as illustrated by experimental data provided herein). As in FIG. 3B platelets showing low PS (PS_{LO}) 320 are designated as Population A containing cells that may optionally be stored for an additional period of time. In repeated step 302, these platelets are returned to storage 150 and treated with CO (by COatm). These platelets are then stored for a further period (step 304). Steps 302 and 304 are thus periodically repeated. Platelets with high PS exposure would be expected to be viable for a much shorter period of storage time. Platelets with high PS exposure would be preferentially used more quickly (that is, after a shorter period of further storage or even immediately. As in FIG. 3B platelets showing high PS (PS_{HI}) 330 are designated and separated as Population B and in step 308 these cells are preferably designated for immediate use in a patient or optionally placed in short-term storage. Thus, low PS exposure platelets are kept in storage until they exhibit high PS exposure and must then be used within a short timeframe, preferably immediately. The separation between PS_{LO} and PS_{HI} of FIG. 3B is essentially at the midpoint between the PS peaks of PS_{LO} and PS_{HI}. In any event, after an extended period of time (optionally between 7-12 days since initial storage) all of the remaining stored platelets will be used or discarded.

Reference is now made to FIG. 4 showing ATP levels in platelets. While under anaerobic COatm the low adenosine triphosphate (ATP) production suffices for Plt survival (5 days). Furthermore the cells do not lose their ability to produce increased ATP upon returning to oxygen containing air (FIG. 4). The results indicate that following storage of 5 days, ATP levels reduced to about a third (28%) of the level in fresh PC. This would be expected considering the strict anaerobic conditions. Nevertheless, the ability to produce higher levels of ATP in the presence of oxygen is reversible as indicated from in the increase back to 2/3 (74%) of the initial value of the ATP of air preserved platelets, following overnight re-aeration.

Testing In Vivo Viability of Stored Platelets:

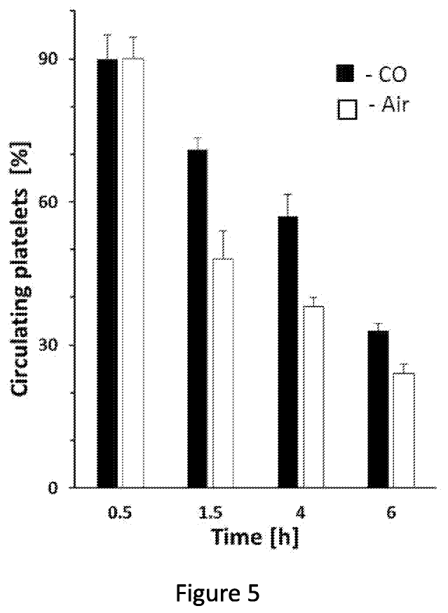

Reference is now made to FIG. 5 showing comparative survival of platelets in vivo after a period of blood circulation. In order to find out whether the platelets stored for longer periods under anaerobic CO atmosphere would survive in vivo following transfusion, their presence in vivo was further measured using the common rabbit model. Fresh human platelets survive in rabbit blood circulation only for hours. The survival of Plt preserved for 9 days under Air- and COatm was tracked. As seen from the results presented in FIG. 5, platelets stored under COatm survived for longer in blood circulation.

Effective Utilization of COatm Preserved Platelets:

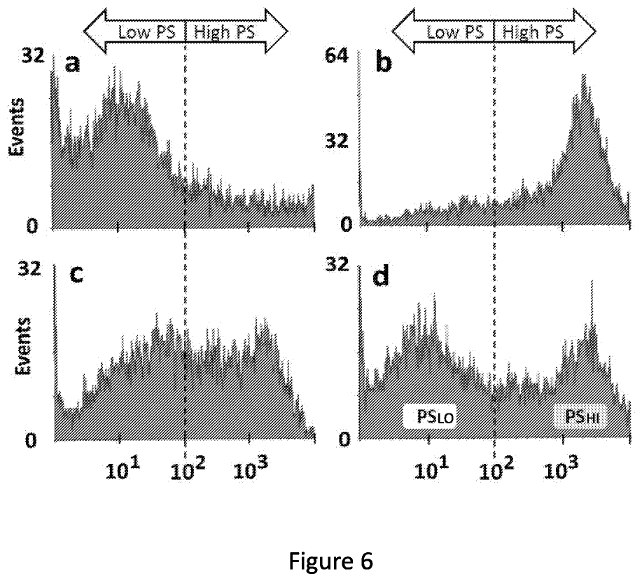

Reference is now made to FIG. 6 showing a comparison of PS exposure in (a) freshly isolated platelets kept under air or (b) COatm for several hours; and, following further storage of 9 days, (c) stored under air, and (d) the platelets atmosphere is exchanged for CO and re-aerated after 9 days. Exposure of PS is a well-known parameter of activation and/or apoptosis in various cells (Liu X M, Chapman G B et al., “Antiapoptotic action of carbon monoxide on cultured vascular smooth muscle cells” Exp Biol Med (Maywood) 2003, 228:572-575; Kim D S, Song L et al., “Carbon Monoxide Inhibits Islet Apoptosis via Induction of Autophagy”, Antioxid Redox Signal 2018, 28:1309-1322). Therefore, it was of importance to find out how PS location is affected in the platelet membrane by COatm. PS exposure of fresh platelets and those stored under Air or COatm were compared.

PS exposure of freshly drawn platelets is shown in FIG. 6a. As seen, most cells have very low PS on the outer surface (note that the scale is logarithmic). Following 9 days of storage, the cells are very heterogeneous, including a larger fraction of cells with PS exposed Plt (FIG. 6c). In the case of COatm-stored platelets, the picture is different: following several hours of storage (FIG. 6b), most cells are PS-exposed. Moreover, there were no further changes during extended storage up to 9 days. However, upon transfer to air (out of CO) (FIG. 6d), a fraction of the 9 days COatm-stored Plt was still PS exposed (PS_{HI}, while another fraction returned to very low PS exposure (PS_{LO}). The peak of the PS_{LO} fraction is the same as that of the fresh Plt. It appears therefore that the PS exposure is reversible for a sub-population which is probably the younger platelets in blood circulation. Thus, the PS_{HI} fraction possibly retains both apoptotic and/or activated Plt.

Based on the above information, COatm-preserved Plt can be divided in two sub-populations according to the PS exposure, using FACS or other technology. As the activity of PS_{HI} activated Plt is lost faster, these should be quickly used for the treatment of acute bleeding, while the PS_{LO} population can be further stored.

The extended storage under CO and subpopulation separation allows efficient utilization of most preserved platelets, each fraction at a different storage time.

Materials and Methods

Preparation of Treated Blood Components

Freshly drawn whole blood was obtained from a human donor under sterile conditions, and stored in gas impermeable bags having a volume of 1.5 times that of the blood volume. The gas environment in the bag atmosphere was then replaced by an atmosphere containing sterile CO by applying a low level vacuum with a water pump of 20 mm Hg. CO was immediately flushed through a 0.25 micron sterile filter. The bag was sealed and agitated for 15 minutes to allow equilibration. This procedure was repeated three times thereby exchanging the atmosphere in the bag and blood with CO. Saturation with CO can be identified in hemoglobin in samples of the treated blood according to typical changes of the light absorption spectrum of the hemoglobin in the visible region by a shift from 577 nm (typical of oxy-hemoglobin) to 569 nm (typical of carbomonoxy-hemoglobin).

The treated blood was kept at room temperature on a shaker until tested (as described in greater detail below) or alternatively until fractionation of the treated blood into blood components (red blood cells, plasma, platelets) using regular blood bank procedures. For further preservation, fractions were separately treated.

Preparation of Platelets

PC fractions were identically prepared from CO pretreated or untreated blood by consecutive centrifugation in a sterile environment using blood bank conditions. Bicarbonate (4% of PC volume) was then added from a stock solution of 750 mM with agitation to yield a final bicarbonate concentration of 30 mM. Next, the PC was treated with CO in a similar manner to whole blood. Alternatively, rather than applying a vacuum, the containers were flushed for 10 min. with sterile CO while agitating the containers, which were then sealed. The containers were allowed to stay at room temperature of 20-24 degree C.° PRP platelets were treated similarly.

Control blood samples were packed under air in the same containers without any additional treatment allowing air transfer. In some experiments an inert gas such as nitrogen was used to exchange the air in the same manner as CO.

ATP Quantitation

A luminometric ATP was measured by Veritas™ Microplate Luminometer (Tuner Biosystems) by using an ATP bioluminescence kit (CLS II) from Roche (cat No. 11 699 709 001) Mannheim Germany The assay was performed according to the manufacturer's instructions. Briefly: 50 μl of cell lysate (500000 cell per sample) or ATP standard concentration was added in advance to each well in 96 well LIA-white plate (Greiner bio one). Automatic injection of 50 μof substrate solution (luciferin and luciferase) was added to each well by the device and the luminescence was measured. ATP concentrations were calculated based on an ATP standard curve. Each sample was tested in triplicate.

In Vivo Platelets Viability Analysis in a Rabbit Model

Female New Zealand white rabbits (2.5-3 kg) were infused with Ethyl Palmytate (EP) to inhibit the Macrophages function (Blajchman M A and Lee D H, “The thrombocytopenic rabbit bleeding time model to evaluate the in vivo hemostatic efficacy of platelets and platelet substitutes”, Transfus Med Rev 1997, 11:95-105). EP solution (10 ml) was prepared by mixing (up to homogeneity) of 2.25 ml EP with 7.75 ml of 5% dextrose containing 1% Tween-20.

The EP-treated rabbits were transfused with PC stored for 7 days under Air or CO. Blood samples at time intervals were analyzed by flow cytometry with FITC labeled anti-CD42a for the presence of human platelets (Rothwell S W, Maglasang P et al., “Survival of fresh human platelets in a rabbit model as traced by flow cytometry”, Transfusion 1998, 38:550-556; Leytin V, Allen D J et al., “A rabbit model for monitoring in vivo viability of human platelet concentrates using flow cytometry”, Transfusion 2002, 42:711-718). 100% represented the total number of normalized human platelet in rabbit circulation after 30 min from the injection.

Analysis of Phosphatidylserine (PS) Exposure by FACS

PS exposure was measured by FACS analysis of bound AnnexinV-FITC using 488 nm for excitation and 530 nm for emission. (Shapira S, Friedman Z et al., “The effect of storage on the expression of platelet membrane phosphatidylserine and the subsequent impact on the coagulant function of stored platelets”, Transfusion 2000, 40:1257-1263)

Results and Discussion

CO Atmosphere Retain Platelet Energy

Under anaerobic conditions, ATP synthesis generally reduces. The ability of metabolic arrested (COatm stored) platelets to recover, should be reflected by ATP synthesis renewal. Therefore, ATP level in COatm stored platelets was measured. As revealed from the results presented in FIG. 4, following 5 days of storage in anaerobic COatm, a significant reduction of ATP level was observed. However, following overnight aeration, the level of ATP was re-elevated, indicating ATP synthesis capability, which is the basis for Plt activity.

Survival of COatm-Stored Platelets In Vivo

For medical use of the preserved platelets, it is of importance to explore if in vitro data directly relate to in vivo conditions. Survival of transfused human platelets in rabbit blood circulation is a model used worldwide for in vivo behavior of stored platelets. (Rothwell SW, Maglasang P et al., “Survival of fresh human platelets in a rabbit model as traced by flow cytometry”, Transfusion 1998, 38:550-556). In rabbit blood circulation human platelets survive only hours and not days but their survival is directly correlated with their fate in the human body.

FIG. 5 demonstrates time dependent survival of 7 days stored human platelets in rabbit blood circulation; air stored in white bars and COatm-stored in black bars. The data shows the percent of platelets left in rabbit blood at several time points. This data demonstrate that COatm-stored platelets survive longer in vivo than air-stored platelets.

Phosphatidylserine (PS) Exposure

The data in FIG. 6c indicate that with time, air stored Platelets undergo heterogeneous exposure of PS, known as an irreversible process (Note the amount PS on the outer leaflet in 4 orders of magnitude extended). By contrast, COatm stored platelets undergo fast and high exposure of PS (FIG. 6b). However, following re-aeration, COatm stored platelets are basically divided into two sub-populations (FIG. 6d):

1) low PS-exposed (similar to fresh, air stored platelets as in FIG. 6a).

2) high PS-exposed (similar to COatm stored platelets as in FIG. 6b).

The low PS-exposed platelets can further be stored for later transfusions. The high PS-exposed platelets include cells which are still useful for treatment of active bleeding and should be immediately used as CO gas has been shown to delay apoptosis (Kim D S, Song L et al., “Carbon Monoxide Inhibits Islet Apoptosis via Induction of Autophagy”, Antioxid Redox Signal 2018, 28:1309-1322).

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present disclosure.

1) A platelet storage device comprising:
a) a core container adapted for extended storage of platelets, wherein said adaptation comprises a COatm inside said core container, wherein said core container is adapted to be closed so that it is gas impermeable; and
b) an outer container for storing said core container, wherein said outer container is adapted to be closed so that it is gas impermeable.

2) (canceled)

3) The platelet storage device of claim 1 wherein said COatm comprises up to 100% carbon monoxide.

4) The platelet storage device of claim 1 wherein said COatm further comprises another gas such as xenon.

5) (canceled)

6) The platelet storage device of claim 1 wherein said outer container comprises an inert gas.

7) The platelet storage device of claim 1 wherein said outer container comprises a CO alarm.

8) A method of storing platelets, comprising:
a) inserting the platelets into a core container, wherein the core container comprises a COatm inside the core container, wherein the core container is adapted to be closed so that it is gas impermeable;
b) treating said platelets by replacing air in said core container with CO and sealing said core container;
c) placing said core container into an outer container wherein the outer container is adapted to be closed so that it is gas impermeable; and
d) sealing said outer container.

9) The method of claim 8 further comprising:
a) opening said outer container;
b) opening said core container to allow escape of said COatm; and
c) illuminating said platelets with a light source for removal of attached CO.

10) The method of claim 8 further comprising, filling said outer container with an inert gas before sealing said outer container.

11) The method of claim 9 further comprising extracting said platelets from said core container.

12) The method of claim 11 further comprising: dividing said extracted platelets into two populations according to at least one characteristic of said platelets correlating with expected viability after further storage, long viability platelets and short viability platelets.

13) The method of claim 12 further comprising: for the long viability platelets:
i) inserting said long viability platelets into said core container;
ii) replacing air in said core container with CO and sealing said core container;
iii) placing said core container into said outer container; and
iv) sealing said outer container.

14) The method of claim 12 further comprising: for the short viability platelets:
immediately using said short viability platelets for transfusion in a patient.

15) The method of claim 12 further comprising: for the short viability platelets: storing said short viability platelets in short-term storage.

16) (canceled)

17) The method of claim 8, wherein said treating step further comprises adding a pH buffering substance to said platelets.

18) The method of claim 17, wherein said pH buffering substance comprises bicarbonate.

19) A method for increasing the yield of stored platelets comprising:
a) storing said platelets in a COatm; and
b) extracting said platelets and separating said platelets into a plurality of populations according to at least one characteristic correlating with expected viability after further storage, long viability platelets and short viability platelets.

20) The method of claim 19, wherein said at least one characteristic relates to PS exposure, such that increased extent of PS exposure correlates with reduced expected viability after further storage.

21) The method of claim 20, wherein said separating is performed by a FACS machine.

22) The method of claim 20 further comprising selecting said short viability platelet population for immediate or rapid use according to said increased extent of PS exposure.

23) The method of claim 20 further comprising selecting said long viability platelet population for further storage in COatm.

24) (canceled)
